(19) United States
(12) Patent Application Publication
(10) Pub. No.: US 2004/0158889 A1
(43) Pub. Date: Aug. 12, 2004

(54) Efficient system for RNA silencing

(76) Inventors: Anna Depicker, Merelbeke (BE); Helena van Houdt, Lochristi (BE)

Correspondence Address: TRASK BRITT, P.O. BOX 2550, SALT LAKE CITY, UT, 84110, US

(21) Appl. No.: 10/813,249

(22) Filed: Mar. 30, 2004

Related U.S. Application Data
(63) Continuation of application No. PCT/EP02/11188, filed on Oct. 2, 2002.

(30) Foreign Application Priority Data
Oct. 5, 2001 (EP) 01203760.2

(51) Int. Cl. A01H 1/00; C12N 15/82

(52) U.S. Cl. 800/278

(57) ABSTRACT

The invention relates to a method for efficient RNA silencing of target genes in eucaryotic cells, particularly plant cells. Consequently, the method can be used to reduce the phenotypic expression of an endogenous gene in a plant cell. Furthermore, the method can be applied in a high throughput screening for mutant phenotypes as a result of RNA silencing of any endogene.

### CROSS-REFERENCE TO RELATED APPLICATION

This application is a continuation of PCT International Patent Application No. PCT/EP/02/11188, filed on Oct. 2, 2002, designating the United States of America, and published, in English, as PCT International Publication No. WO 03/031632 A1 on Apr. 17, 2003, the contents of the entirety of which is incorporated by this reference.

### TECHNICAL FIELD

The invention relates generally to biotechnology, and more particularly to a method for efficient RNA silencing in eucaryotic cells, particularly plant cells. Consequently, the method can be used to reduce the phenotypic expression of an endogenous gene in a plant cell. Furthermore, the method can be applied in a high throughput screening for RNA silencing.

### BACKGROUND

“RNA silencing” is a type of gene regulation based on sequence-specific targeting and degradation of RNA. The term encompasses related pathways found in a broad range of eukaryotic organisms, including fungi, plants, and animals.

In plants, RNA silencing serves as an antiviral defense and many plant viruses encode suppressors of silencing. Also, it becomes clear that elements of the RNA silencing system are essential for gene regulation in development. The emerging view is that RNA silencing is part of a sophisticated network of interconnected pathways for cellular defense, transposon surveillance, and regulation of development. Based on the sequence specific RNA degradation, RNA silencing has become a powerful tool to manipulate gene expression experimentally. RNA silencing was first discovered in transgenic plants, where it was termed co-suppression or posttranscriptional gene silencing (PTGS). Sequence-specific RNA degradation processes related to PTGS have also been found in ciliates, fungi, and a variety of animals from Caenorhabditis elegans to mice (RNA interference).

A key feature uniting the RNA silencing pathways in different organisms is the importance of double-stranded RNA (dsRNA) as a trigger or an intermediate. The dsRNA is cleaved into small interfering RNAs (21 to 25 nucleotides) of both polarities, and these are thought to act as guides to direct the RNA degradation machinery to the target RNAs. An intriguing aspect of RNA silencing in plants is that it can be triggered locally and then spread via a mobile silencing signal. In plants, RNA silencing is correlated with methylation of homologous transgene DNA in the nucleus. Other types of epigenetic modifications may be associated with silencing in other organisms.

It is known from the art that transgenes encoding ds or self-complementary (hairpin) RNAs of endogenous gene sequences are highly effective at directing the cell's degradation mechanism against endogenous (ss) mRNAs, thus giving targeted gene suppression. This discovery has enabled the transgenic enhancement of a plant's defense mechanism against viruses that it is unable to combat unaided. It has also shed light on how antisense and co-suppression might operate: by the inadvertent integration of two copies of the transgenes in an inverted repeat orientation, such that read-through transcription from one gene into the adjacent copy produces RNA with self-complementary sequences.

RNA silencing is induced in plants by transgenes designed to produce either sense or antisense transcripts. Furthermore, transgenes engineered to produce self-complementary transcripts (dsRNAs) are potent and consistent inducers of RNA silencing. Finally, replication of plant viruses, many of which produce dsRNA replication intermediates, causes a type of RNA silencing called Virus Induced Gene Silencing (VIGS). Whether VIGS, and the different types of transgene-induced RNA silencing in plants result from similar or distinct mechanisms is still a matter of debate. However, recent genetic evidence raises the possibility that the RNA silencing pathway is branched and that the branches converge in the production of dsRNA.

### SUMMARY OF THE INVENTION

Until recently, RNA silencing was viewed primarily as a thorn in the side of plant molecular geneticists, limiting expression of transgenes and interfering with a number of applications that require consistent, high-level transgene expression. With our present understanding of the process, however, it is clear that RNA silencing could have enormous potential for engineering control of gene expression, as well as for the use as a tool in functional genomics. It could be experimentally induced and targeted to a single specific gene or even to a family of related genes. Likewise, ds RNA-induced TGS may have similar potential to control gene expression. Although methods for RNA silencing have been described in the art (e.g., WO99/53050, WO99/32619, WO99/61632, and WO98/53083), a need exists to develop alternative and more efficient tools for RNA silencing.

In the present invention, we have developed a highly efficient method for RNA silencing that can also be used as a tool for high throughput silencing. The method uses a host that carries already a silenced locus and a second recombinant gene comprising a region that is homologous with the silenced locus. Although it is known that the recombinant gene will be silenced, we have surprisingly found that also target genes, which have no significant homology with the silenced locus but have homology with the recombinant gene, are efficiently silenced.

The present invention deals with an efficient method for RNA silencing in a eucaryotic host. The method makes use of a host that already comprises a silenced locus. Such a silenced locus can for example be generated by methods known in the art. For example the publication of De Buck and Depicker, 2001 and other publications, and also PCT patent publications WO99/53050, WO99/32619, WO99/61632, and WO98/53083 describe methods to obtain RNA silencing and for generating a silenced recombinant locus. The ‘target gene’ is here defined as the gene of interest for silencing or to down-regulate its expression. An important aspect of this invention is that the target gene has no significant homology with the silenced locus. No significant homology means that either the overall homology is less than 40, 35, 30, 25% or even less or that no contiguous stretch of at least 23 identical nucleotides are present (Thomas et al., 2001). Homology is typically measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various insertions, deletions, substitutions, and other modifications. Silencing of the target gene in the present invention occurs via an intermediate step and hence our method is designated as domino silencing (FIG. 1). In the intermediate step a recombinant gene construct is introduced by transformation into the host comprising the silenced locus. The recombinant gene construct has a region of homology with the silenced locus already present. The region of homology is preferably more than 60, 70, 80, 90, 95 or even more than 99% homologous. The homologous region between the silenced locus and the recombinant gene can be found in the 5′ untranslated or 3′ untranslated region of the recombinant gene construct. Furthermore, the recombinant gene construct has a region of minimal 23 nucleotides (Thomas et al., 2001), but preferably longer, that are identical with the target gene, or has a region of overall homology of more than 60, 70, 80, 90, 95 or even more than 99%. A recombinant gene is defined herein as a construct which does not naturally occur in nature. A non-limiting example of a recombinant gene construct is a construct wherein the coding region of a gene is operably linked to a 5′ untranslated region and/or to a 3′ untranslated region of one or more other genes, alternatively the 5′ or 3′ untranslated region is an artificial sequence.

Thus, in one embodiment the invention provides a method for obtaining efficient RNA silencing of a target gene comprising the introduction of a recombinant gene into a host that comprises a silenced locus and an unsilenced target gene whereby the recombinant gene comprises a region that is homologous with the silenced locus and whereby the target gene has homology with the recombinant gene but has no significant homology with the silenced locus.

In another embodiment, the method is used wherein the host is a plant or plant cell.

In another embodiment, the method of the invention can be used for high throughput gene silencing. Indeed, a recombinant gene library can be made wherein for example every gene or coding region thereof is combined with (operably linked with) a region of homology with the silenced gene that resides in the silenced locus and the recombinant gene library can be transformed to an eukaryotic host or individual (specific) genes derived from the recombinant gene library can be transformed into an eukaryotic host wherein silencing of specific genes is wanted.

In yet another embodiment, the invention provides a plant or plant cell that comprises a silenced locus and wherein a silenced target gene is obtained through the introduction of a recombinant gene according to the current method of the invention.

In yet another embodiment, the RNA silencing of the target gene is obtained in more than 80, 85, 90 or 95% of the transgenic organisms.

In yet another embodiment, the RNA silencing of the target gene occurs at an efficiency of more than 80, 85, 90 or 95% as compared to the level of the unsilenced expression of the target gene.

### BRIEF DESCRIPTION OF THE SEVERAL VIEWS OF THE DRAWINGS

FIG. 1: Schematic outline of homology between a silenced locus X, a recombinant gene Y and a target gene Z.

FIG. 2: Schematic outline of the T-DNA constructs that are present in silenced locus X₁, recombinant gene Y₁ and target gene Z₁ (T-DNAs of pGVCHS287, pGUSchsS and pXD610 respectively) and of the transcript homology between X₁, Y₁ and Z₁.

LB and RB: left and right T-DNA border respectively; Pnos: nopaline synthase promoter; hpt: hygromycin phosphotransferase coding sequence; 3′nos: 3′untranslated region of the nopaline synthase gene; P35S; Cauliflower mosaic virus 35S promoter; nptII c.s., neomycin phosphotransferase II coding sequence; 3′chs: 3′untranslated region of the chalcone synthase gene of Anthirrinum majus; +1: transcription start; Aₙ: poly A-tail; gus c.s.: β-glucuronidase coding sequence; Pss: promoter of the small subunit of rubisco; bar: phosphinotricine transferase coding sequence; 3′g7: 3′untranslated region of the Agrobacterium octopine T-DNA gene 7; 3′ocs: 3′untranslated region of octopine synthase gene.

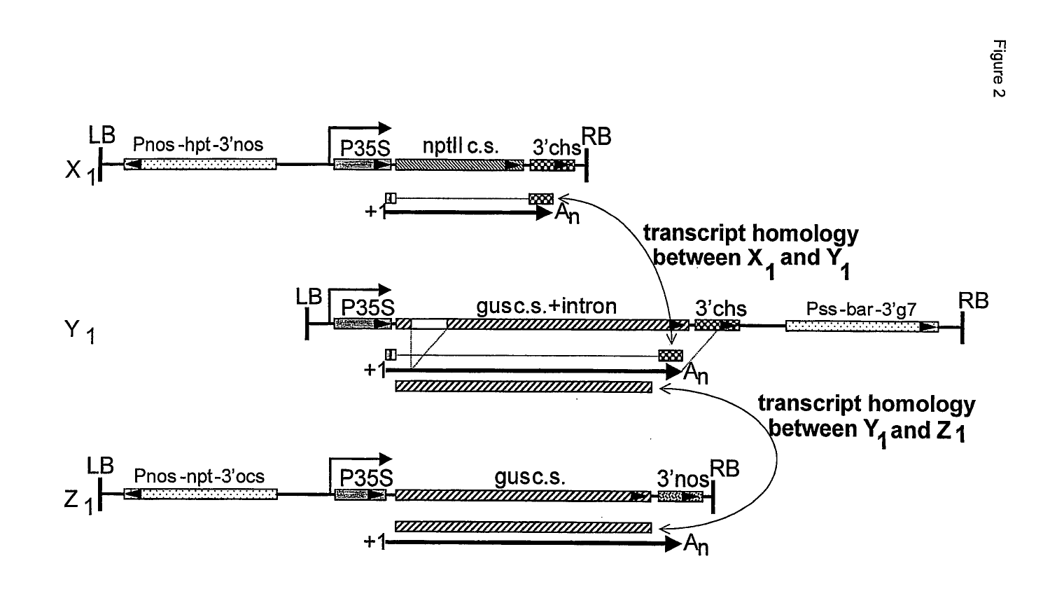

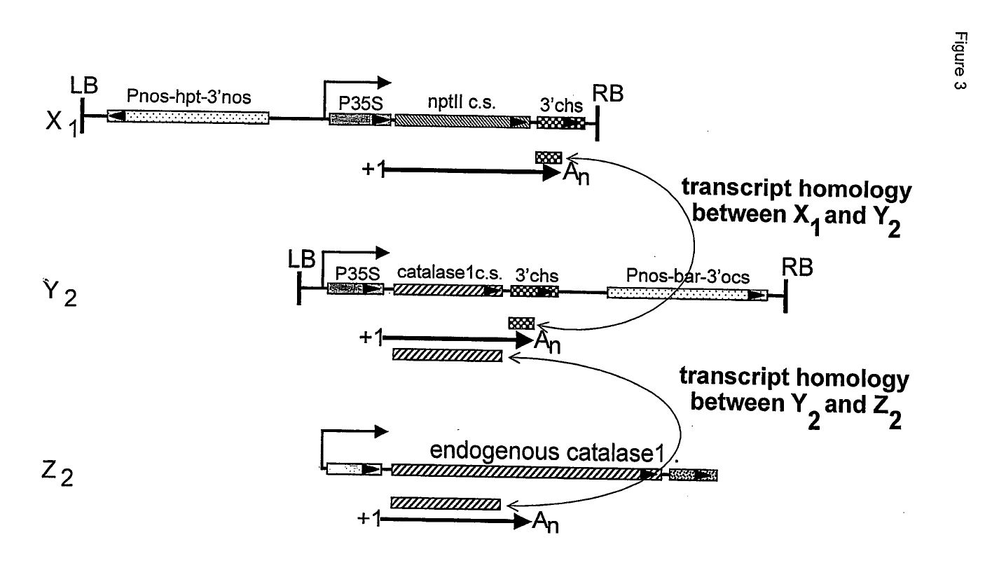

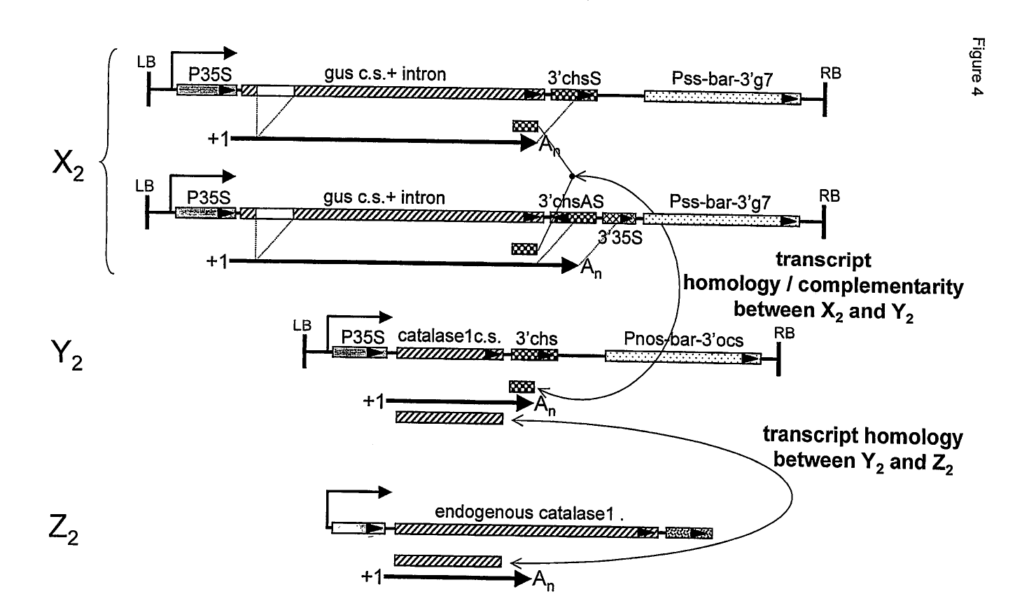

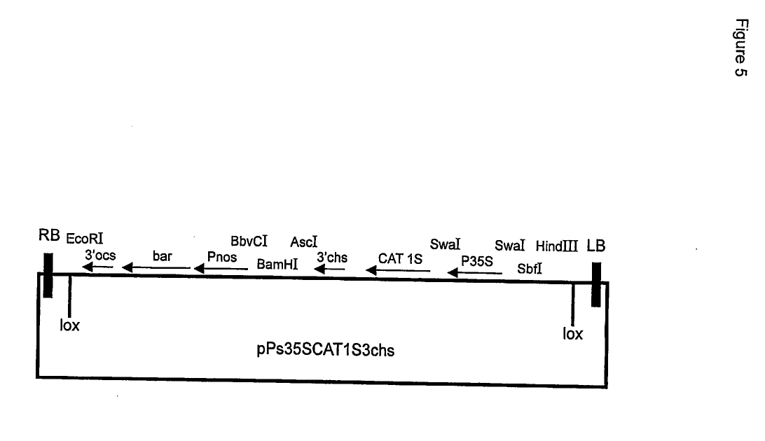

FIG. 3: Schematic outline of the T-DNA construct present in silenced locus X₁ and of the transiently introduced T-DNAs Y₂ (T-DNAs of pGVCHS287 and pPs35SCAT1S3chs, respectively) and of the transcript homology between X₁, Y₂ and Z₂ (the catalase1 endogene). Abbreviations as in FIG. 2

FIG. 4: Schematic outline of the T-DNA constructs present in silenced locus X₂ and of the transiently introduced T-DNAs Y₂ (T-DNAs of pGUSchsS+pGUSchsAS, and pPs35SCAT1S3chs, respectively) and of the transcript homology between X₂, Y₂ and Z₂ (the catalase1 endogene). Abbreviations as in FIG. 2

FIG. 5: pPs35SCAT1S3chs

### DETAILED DESCRIPTION OF THE INVENTION

A post-transcriptionally silenced inverted repeat transgene locus can trigger silencing of a reporter gene producing non-homologous transcripts.

We studied the interaction between three transgene loci X₁, Y₁ and Z₁ (FIG. 2. For a detailed description of all loci and constructs, see materials and methods) to address the question whether or not a stepwise homology between loci can lead to silencing.

It has been demonstrated previously that the post-transcriptionally silenced nptII genes in locus XI are capable to in trans silence transiently expressed genes with partial transcript homology to their nptII transcripts (Van Houdt et al., 2000 b). We subsequently found that also a stably expressed β-glucuronidase (gus) gene (in locus Y₁), with partial transcript homology to the nptII transcripts of the silencing inducing locus X₁, becomes efficiently silenced in trans (FIG. 2: X₁ and Y₁ and table 1: X₁Y₁ compared to Y₁). On the contrary, the nptII genes of locus X₁ are not able to trigger silencing of the gus genes in locus Z₁ which is expected as the genes of both loci produce transcripts without significant homology (FIG. 2). The homology between the two transcripts of X₁ and Y₁ is mainly situated in the 3′untranslated region (250 nucleotides), but also the 5′untranslated sequences show a small region of homology (29 nucleotides). These results demonstrate that the in trans silencing effects are not triggered by promoter homology. When Y₁ and Z₁ loci are combined in so called Y₁Z₁ hybrids both types of gus genes, having transcript homology in the gus coding sequence of 1809 nucleotides, remain highly expressed as reflected in the normal gus activity showing that the RNA silencing mechanism does not become activated (Table 1: Y₁Z₁ compared to Y₁ and Z₁). Surprisingly, upon creation of a stepwise homology between X₁ and Z₁ by introducing locus Y₁, the new observation described here is that also the gus expression in locus Z₁ is reduced in X₁Y₁Z₁ plants (Table 1: X₁Y₁Z₁ compared to Y₁Z₁). Thus, creating a stepwise homology between a silenced locus and a target gene by introducing a recombinant gene is sufficient to trigger silencing of the target.

Silencing inducing transgene loci can trigger silencing of a non-homologous endogene.

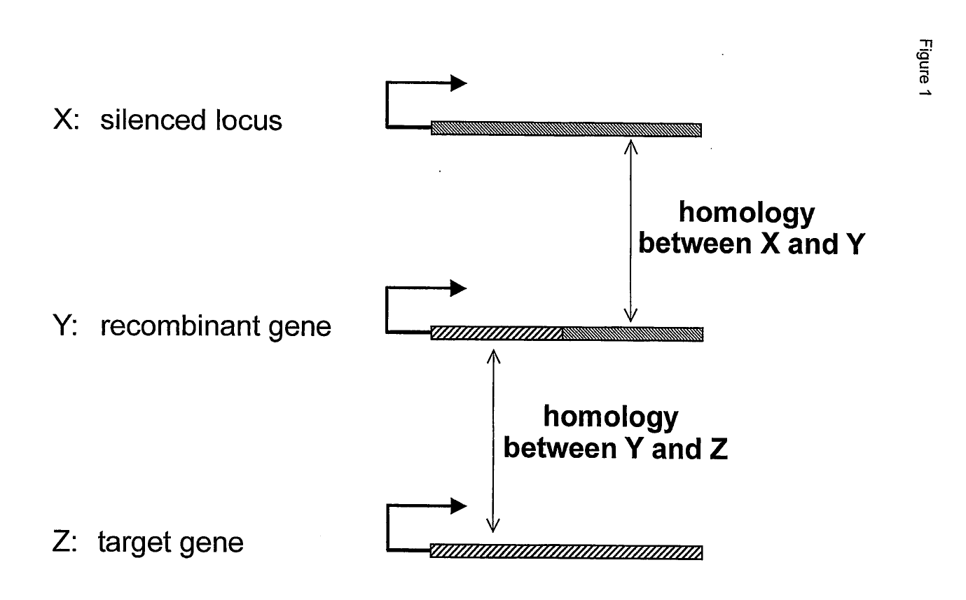

We further assessed the universality and the usefulness in high throughput functional gene analyses of silencing elicited by a stepwise homology in trans, called domino silencing. Therefore, we evaluated whether the expression of the tobacco endogenous catalase1 (cat1) genes is reduced in plants carrying a silencing locus (X locus) showing no significant homology with the catalase endogene by introducing a recombinant gene (Y construct). As silencing locus we used either X₁ or X₂ (FIG. 2: locus X₁, FIG. 3: locus X₂), in either case containing the 3′ chalcone synthase sequences of Anthirrinum majus (3′chs). As transmitter for silencing we constructed a recombinant gene composed of the catalase1 coding sequence and the 3′ chs region under control of the 35S promoter (P35S) (residing on T-DNA pPs35SCAT1S3chs, FIGS. 2 and 3: T-DNA in Y₂). The recombinant cat1 3′chs genes (Y2) were introduced in tobacco leaves bearing locus X₁ (or X₂) via Agrobacterium injection. As a negative control, we introduced a recombinant gene in which the cat1 coding sequence is replaced by the gus coding sequence (pGUSchsS, T-DNA construct as in locus Y₁ FIG. 1). In this case, no stepwise homology is created between the silencing inducing locus and the target catalase endogenes. As a positive control, the recombinant construct Y₂ was also introduced in transgenic tobacco with silenced catalase1 genes by the presence of a catalase1 antisense construct (Cat1AS in Champnongpol et al., 1996). Sixteen days after Agrobacterium injection, the catalase activity was determined in protein extracts of injected leaf tissue and compared with the activity in non-injected wild type (SR1) leaf tissue (Table 2). The results indicate that domino silencing is also applicable to endogenes since the catalase activity is clearly reduced in 6 out of 7 samples, while it remains high in the negative controls. In conclusion, not only an inverted repeat-bearing silencing-inducing transgene locus, but also a silencing-inducing locus in which the two residing chimeric genes give rise to transcripts with complementarity in the 3′UTR (3′chs)(FIG. 3: X₂), is able to trigger domino silencing reducing endogenous catalase expression.

TABLE 1
Results of a GUS-activity determination in protein extracts of leaf
tissue harvested from tobacco plants containing different
combinations of the loci X₁, Y₁ and Z₁
(FIG. 2). The mean values of a number of plants (n) are given

| | GUS-act.¹ 4 weeks² | | GUS-act. Mature² | |
|---|---|---|---|---|
| Genotype | U GUS/mg TSP | N | U GUS/mg TSP | n |
| X₁ | <³ | 1 | < | 1 |
| Y₁ | 368 ± 165⁴ | 9 | n.d. | — |
| Z₁ | 126 ± 30 | 10 | 48 ± 8 | 5 |
| X₁Y₁ | 2 ± 1 | 4 | 4 ± 2 | 4 |
| X₁Z₁ | 139 ± 35 | 9 | 46 ± 14 | 5 |
| Y₁Z₁ | 477 ± 101 | 10 | 231 ± 106 | 6 |
| X₁Y₁Z₁⁵ → Y₁Z₁ → | 195 ± 104 | 16 | 315 ± 46 | 8 |
| X₁Y₁Z₁ | 4 ± 3 | 22 | 12 ± 4 | 9 |

¹The mean GUS-activity (GUS-act.) was calculated, using n samples and expressed as units (U) GUS per milligram of total soluble protein (TSP).
²The plants were analyzed in two different developmental stages; 4 weeks after sowing and at a mature stage just before onset of flowering.
³below detection limit (1 U GUS/mg TSP)
⁴standard deviation
⁵Growth of X₁Y₁Z₁ plants was performed in conditions that both Y₁Z₁ and X₁Y₁Z₁ plants were able to develop. A PCR screen with X₁₋specific primers was performed to discriminate between presence and absence of X₁.
n.d. not determined

TABLE 2
Results of a catalase-activity determination in protein
extracts of leaf tissue harvested from Agrobacterium
injected tobacco leaves.

| Genotype injected | Construct introduced via | catalase activity 16 days | |
|---|---|---|---|
| Plant | Agrobacterium injection | after injection (60 μg TSP) | |
| WT (SR1) | -(non-injected) | −0.2116² | 100%³ |
| X₁ | PGUSchsS | −0.2556 | 121% |
| X₁ | Y₂ | −0.0589 | 27% |
| X₁⁴ | Y₂ | −0.0698 | 33% |
| X₂ | PGUSchsS | −0.1782 | 84% |
| X₂ | Y₂ | −0.0641 | 30% |
| X₂ | Y₂ | −0.0987 | 47% |
| X₂⁴ | Y₂ | −0.0914 | 43% |
| X₂⁴ | Y₂ | −0.1996 | 94% |
| X₂⁴ | Y₂ | −0.0627 | 30% |
| Cat1AS | Y₂ | −0.0439 | 21% |

¹X₁, see FIG. 3; X₂, see FIG. 4.
²The mean of two samples independently measured (−0.2270 and −0.1963).
³The catalase activity in wild type SR1 tobacco leaves was set to 100%.
⁴24 hours after Agrobacterium injection, the plants were placed under high light conditions for 24 hours (1000 μmol/m² s). This treatment is known to stimulate endogenous catalase 1 transcription. As the degree of cat suppression is similar in uninduced as in induced situation, the data indicate that enhanced transcription of the endogenous catalase target is not required to trigger domino silencing.

### EXAMPLES

### Materials and Methods

Plasmid Construction

pPs35SCAT1S3chs: The T-DNA of this plasmid is schematically shown in FIG. 3: Y₂ and the nucleotide sequence is depicted in SEQ ID NO:1 of the accompanying and incorporated herein SEQUENCE LISTING.

Description of the Transgene Loci and Production of Hybrid Plants

Locus X₁ harbors an inverted repeat about the right T-DNA border of construct pGVCHS287, carrying a neomycin phosphotransferase II (nptII) gene under the control of the Cauliflower mosaic virus 35S promoter (P35S) and the 3′signalling sequences of the Anthirrinum majus chalcone synthase gene (3′chs). The nptII genes are post-transcriptionally silenced and can trigger in trans silencing and methylation of homologous target genes (Van Houdt et al., 2000 a and b and FIG. 2).

Locus Y₁ contains a single copy of the pGUSchsS T-DNA, containing a gus gene under the control of P35S and 3′chs (in transformant GUSchsS29) and shows normal levels of gus expression (FIG. 2).

Locus Z₁ contains more than one copy of the pXD610 T-DNA, harboring the gus gene under control of P35S and the 3′untranslated region (UTR) of the nopaline synthase gene (3′nos), (in plant LXD610-2) and shows normal gus expression (De Loose et al., 1995 and FIG. 2).

Locus X₂ contains a single copy of both the pGUSchsS and pGUSchsAS T-DNA (in transformant GUSchsS+GUSchsAS11) and triggers silencing in cis of the gus genes, but also in trans of (partially) homologous genes (FIG. 4).

X₁ and Z₁ hemizygous plants were obtained as hybrid progeny of the crossing of tobacco plants homozygous for locus X₁ (=Holo1; Van Houdt et al., 2000 a and b) and homozygous for locus Z₁ (=LXD610-2/9 De Loose et al., 1995) to wild type SR1 respectively. Y₁ hemizygous plants were obtained by crossing the hemizygous primary tobacco transformant GUSchsS29 to SR1 and selecting for the presence of locus Y₁ in the hybrid progeny. X₁Y₁ and Y₁Z₁ hemizygous plants are the hybrid progeny plants of the cross between Holo1 and GUSchsS29 and between GUSchsS29 and LXD610-2/9 respectively that are selected for the presence of Y₁. X₁Z₁ hemizygous plants are the hybrid progeny of the cross between Holo1 and LXD610-2/9. X₁Y₁Z₁ hemizygous plants were obtained by crossing X₁Y₁ hemizygous plants to LXD610-2/9; as we only selected for the presence of Y₁ in the hybrid progeny both Y₁Z₁ and X₁Y₁Z₁ hemizygous plants were obtained.

Preparation of Agrobacteria and Injection

The Agrobacteria C58C1Rif^{R} (pGV2260) (pGUSchsS)Cb^{R},PPT^{R} or C58C1Rif^{R}(pMP90)(pPs35SCAT1S3chs)Gm^{R},PPT^{R} were mainly grown as described by Kapila et al., 1997 except that the Agrobacteria were resuspended in MMA to a final OD₆₀₀ of 1. Greenhouse grown plants of 10 to 15 cm in height were used. Half of the third top leaf was injected via the lower surface using a 5 ml syringe while the leaf remained attached to the plant. The plants were kept in the greenhouse and 16 days after injection three to four discs of 11 mm in diameter were excised from the injected tissue for the preparation of a fresh protein extract to determine the catalase activity.

Enzymatic Assays

Preparation of the protein extracts and GUS-activity measurements were done as previously described (Van Houdt et al., 2000 b). Preparation of the protein extracts for catalase-activity measurement and the spectrophotometric catalase-activity determination was done according to Champnongpol et al., 1996.

### REFERENCES

Van Houdt, H., Kovarik, A., Van Montagu, M., and Depicker, A. (2000 a). Cross-talk between posttranscriptionally silenced neomycin phosphotransferase II transgenes. FEBS Lett. 467, 41-46.

Van Houdt, H., Kovarik, A., Van Montagu, M., and Depicker, A. (2000 b) Both sense and antisense RNAs are targets for the sense transgene-induced posttranscriptional silencing mechanism. Mol. Gen. Genet. 263, 995-1002.

De Loose, M., Danthinne, X., Van Bockstaele, E., Van Montagu, M. and Depicker, A., (1995) Different 5′leader sequences modulate β-glucuronidase accumulation levels in transgenic Nicotiana tobacum plants. Euphytica 85, 209-216.

Kapila, J., De Rycke, R., Van Montagu, M. and Angenon, G. (1997) An Agrobacterium-mediated transient gene expression system for intact leaves. Plant Science 122, 101-108.

Champnongpol, S., Willekens, H., Langebartels, C., Van Montagu, M., Inzé, D., and Van Camp, W. (1996) Transgenic tobacco with a reduced catalase activity develops necrotic lesions and induces pathogenesis-related expression under high light. Plant J. 10(3), 491-503.

Thomas, C. L., Jones, L., Baulcombe, D. C. and Maule, A. J. (2001) Size constraints for targetting post-transcriptional gene silencing and for RNA-directed methylation in Nicotiana benthamiana using potato virus X vector. Plant J. 25(4), 417-425.

De Buck, S. and Depicker, A. (2001) Disruption of their palindromic arrangement leads to selective loss of DNA methylation in inversely repeated gus transgenes in Arabidopsis. Mol. Gen. Genom. 265, 1060-1068.

What is claimed is:

1. A method for obtaining efficient RNA silencing of a target gene comprising:
introducing a recombinant gene into a host comprising a silenced locus and the target gene, wherein
the recombinant gene comprises a region homologous with the silenced locus and
the target gene has homology with the recombinant gene, but has no significant homology with the silenced locus,
thus RNA silencing the target gene.

2. The method according to claim 1 wherein the host comprises a plant cell.

3. The method according to claim 1 wherein the RNA silencing of the target gene is obtained in more than 95% of the hosts.

4. The method according to claim 2 wherein the RNA silencing of the target gene is obtained in more than 95% of the hosts.

5. The method according to claim 1 wherein RNA silencing of the target gene is obtained in more than 85% of the hosts.

6. The method according to claim 2 wherein RNA silencing of the target gene is obtained in more than 85% of the hosts.

7. The method according to claim 1 wherein the RNA silencing of the target gene occurs at an efficiency of more than 95% as compared to the level of the unsilenced expression of the target gene.

8. The method according to claim 2 wherein the RNA silencing of the target gene occurs at an efficiency of more than 95% as compared to the level of the unsilenced expression of the target gene.

9. The method according to claim 1 wherein the RNA silencing of the target gene occurs at an efficiency of more than 85% as compared to the level of the unsilenced expression of the target gene.

10. The method according to claim 2 wherein the RNA silencing of the target gene occurs at an efficiency of more than 85% as compared to the level of the unsilenced expression of the target gene.

11. The method according to claim 1 to obtain high throughput gene silencing.

12. The method according to claim 2 to obtain high throughput gene silencing.

13. A plant or plant cell comprising a silenced target gene obtainable by the method according to claim 1.


SEQUENCE LISTING (1 sequence)

SEQ ID NO: 1 - DNA, 10635 bp, Artificial Sequence
  pPs35SCAT1S3chs
agattcgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgcgacaaattacaacggtatatatcctgccagtactcggccgtcgaataacttcgtataatgtatgctatacgaagttatgaattcgcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagctggaagatttgatggatcctcatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatctgtttaaacgttaacggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaatcgttgcggttctgtcagttccaaacgtaaaacggcttgtcccgcgtcatcggcgggggtcataacgtgactcccttaattctccgctcatgatcaagctacctcagcaggatccggcgcgccatggtcgataagaaaaggcaatttgtagatgttaattcataacatctcctccatgacttaaaaaacttgcaaaagatttatatagaaatacttaaatattttgactaaaaaaaaaaaaaaaaaaacacacacataaaccaacaaataacataaattatttttatatagcctttatttcaatgatcacaacgaaacaatacaagtacaaagcgttacaagagagaaatcgccaatatagctcacatgcagcacacatcacaataataggtaaccatgtccacttttttattacggaaataagaaaataacccaacccccgtacccgggttcatatgcttggtctcacattaagcctagaagctagcttttgacccagagatttgtcagcctgagaccagtatgagatccaaatgctgcggatctcataagtgatacgaggatcagacaaggtctccacccaccgacgaataaagcgttcttgcctgtctggtgtgaatgagcggtacctttctcctggttgcttgaaattgttctctttctgaatgacacacttctcgcgtttgccagtgcacattgtagaaggaataggatacttctcagcatggcgaacaggatcataccttgaagggaagtagtcgatctcctcatccctgtgcataaaattcatggagccatcgtagtgattgttgtgatgagcgcattttggagcattagcaggtagttgcaaatagtttggtccaagtcgatacctctgggtatcagagtaggagaaaatacgagtttgaagcatcttatcatctgagtaataaacccctggaacaacaatagaagggcagaaagctagctgctcattctcattagagaagttatcaatgttcttgttcagaactaatcttcccaccggctgcaaaggcaagatatcctctggccaagtttttgtcacatcaagtggatcaaaatcaaatctgtcttcatgatctggatccatagtccccgggcagtgggcgatttgatttaaatctctagaatagtaaattgtaatgttgtttgttgtttgttttgttgtggtaattgttgtaaaaatacggatcgtcctgcagtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacgaagattttcttcttgtcattgagtcgtaaaagactctgtatgaactgttcgccagtcttcacggcgagttctgttagatcctcgatctgaatttttgactccatggcctttgattcagtaggaactactttcttagagactccaatctctattacttgccttggtttatgaagcaagccttgaatcgtccatactggaatagtacttctgatcttgagaaatatatctttctctgtgttcttgatgcagttagtcctgaatcttttgactgcatctttaaccttcttgggaaggtatttgatctcctggagattattactcgggtagatcgtcttgatgagacctgccgcgtaggcctctctaaccatctgtgggtcagcattctttctgaaattgaagaggctaatcttctcattatcggtggtgaacatggtatcgtcaccttctccgtcgaactttcttcctagatcgtagagatagagaaagtcgtccatggtgatctccggggcaaaggagatctctagagtcgagatttaaatcctaaatcctgcaggaagcttaccggtataacttcgtatagcatacattatacgaagttatccatggagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacggtaccggaacgaccaagcaagctagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaagtgctgtgcacggatctgccctggcttcaggagatcggaagacctcggccgtccgggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcagggatcgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccggaaaacgcaagcgcaaagagaaagcaggtagcttgcagtgggcttacatggcgatagctagactgggcggttttatggacagcaagcgaaccggaattgcc